(19) United States
(12) Patent Application Publication
(10) Pub. No.: US 2007/0073410 A1
(43) Pub. Date: Mar. 29, 2007

(54) Flexible prosthetic cup

(75) Inventor: Patrick Raugel, Ramsey, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/523,488

(22) Filed: Sep. 19, 2006

(30) Foreign Application Priority Data
Sep. 23, 2005 (GB) GB-A-05 19490.7

(51) Int. Cl. A61F 2/32; A61F 2/34

(52) U.S. Cl. 623/22.18; 623/22.3; 623/23.43

(57) ABSTRACT

A prosthetic flexible bearing element cup or cup bearing liner is adapted for use with a part-spherical bearing head. The flexible bearing element includes an inner bearing surface which has a first portion which is substantially part-spherical about a hemispherical center located on a main loading axis, and a second portion at least part of which is relieved with respect to the first portion. The relieved part of the second portion has a radial distance (d) from the hemispherical center which increases from an inner end towards an outer end as the angle of rotation (θ) about the hemispherical center increases towards the rim of the cup.

### CROSS-REFERENCE TO RELATED APPLICATIONS

The present application claims the benefit of Great Britain Patent Application No. GB 0519490.7, filed Sep. 23, 2005, entitled Prosthetic Joints, the disclosure of which is hereby incorporated herein by reference.

### BACKGROUND OF THE INVENTION

This invention relates to prosthetic joints and more particularly to a prosthetic joint which includes a part-spherical femoral bearing head and a flexible bearing element cup or cup bearing liner adapted for use with a bearing head such as in a total hip joint.

Flexible bearing element cups and cup bearing liners are known which include an inner bearing surface which has a first portion which is substantially part-spherical about a hemispherical center and in which there is a polar bearing area, and a second portion which is relieved with respect to the first portion and which has a radial distance from the hemispherical center which increases as the angle of rotation about the hemispherical curve increases towards the rim of the cup, the second portion being tangential to the first in a transition portion.

Another construction of this type is shown in United States Patent Publication No. 2005/0256584. The invention in this Application is directed to the curvature of the relieved portion of the cup and the various parameters set out in the Patent are said to create a shape of the non-spherical portion which has the configuration of part of an involute. The concept is defined by regarding the involute as being formed by a thread which is unwould from an imaginary bobbin which is positioned at its circumference on the center of the sphere which defines the spherical portion of the cup.

Cups of the kind referred to which have a relieved portion with respect to the first hemispherical portion of the cup are employed for various reasons. It is desirable to reduce the contact area of the ball with the cup when reaching extreme angles as this tends to cause friction between them and the relieving alleviates this. Again, the flexibility of the cup may require the relieving to take place and to prevent seizure.

It has been found from experimentation that the amount and shape of the relieving can be varied according to the requirements.

Another consideration is that the fit of the ball in the bearing surface can be closer with a small head ball than with a larger head ball. It is also dependent upon the amount of lubrication in the joint so that amount of relieving must be adjusted appropriately. The amount of stiffness in the cup or liner effects its deformation.

### SUMMARY OF THE INVENTION

According to the present invention a prosthetic flexible bearing element cup or cup bearing liner adapted for use with a part-spherical bearing head includes an inner bearing surface which has a first portion which is substantially part-spherical about a hemispherical center located on a main loading axis, and a second portion at least part of which is relieved with respect to the first portion and which over the relieved part has a radial distance (d) from the hemispherical center which increases from an inner end towards an outer end as the angle of rotation (θ) about the hemispherical center increases towards the rim of the cup. The distance of the relieved part of the surface of the second portion from a line representing the shape of the outer surface of the part-spherical bearing head when located on said main loading axis and bearing against said first portion of the inner bearing surface at any given radial distance (d) is determined by the formula
d=a(e^{θ ln b}−1)
where
D is the radial distance in millimeters between the line representing said shape of the outer surface of the part-spherical bearing head when located on said first portion of the inner bearing surface and with its center located on said hemispherical center and the relieved part of the second portion
θ is the angle in degrees about the hemispherical center, and
a and b are values determined by selecting angle an (θ₁) and a distance (d₁) at or towards said inner end of the second portion and placing them in said formula, selecting an angle (θ₂) and a distance (d₂ at or towards the outer end of second portion adjacent the rim and placing them in the formula and solving the two equations together.

Thus, for varying angles around the length of the second portion, a series of distances (d) can be calculated and which define the shape of the relieving in relation to the line representing said bearing head when in position.

It has been found that the use of the values of a and b in the formula can provide desired shapes of curvature of the relieved portion and which depend upon the angle in degrees about the hemispherical center and are suitable for use for various requirements. The particular shape for a particular requirement can be found by experimentation.

Preferred successful values of a and b are as set out in the following Table:

| a | b |
|---|---|
| 8.2 | 1.001 |
| 1.25 | 1.005 |
| 0.43 | 1.01 |
| 0.305 | 1.012 |
| 0.19 | 1.015 |
| 0.092 | 1.02 |
| 0.046 | 1.025 |
| 0.024 | 1.03 |
| 0.0125 | 1.035 |
| 0.0068 | 1.04 |
| 0.00035 | 1.045 |
| 0.0019 | 1.05 |
| 0.00054 | 1.06 |
| 0.00015 | 1.07 |
| 0.000004 | 1.1 |

Curves with the shapes defined by these numbers have been found to meet most requirements. Preferably the second portion extends from the main loading axis to the end thereof adjacent its rim.

If desired a transition portion of the inner bearing surface between the first portion and the relieved part of the second portion can be provided. Thus the end of the second portion can overlap the first portion up to the main loading axis, to provide said transition portion.

The inner bearing surface can be formed in a one-piece cup made from, for example, metal, a synthetic plastics material, a combination of synthetic materials and fibres, or a ceramic material.

In an alternative construction the inner bearing surface can be formed on a bearing liner carried In a backing and the liner can be made from metal, a synthetic plastics material, a combination of synthetic material and fibres or a ceramic material, the backing can, for example, be made from metal, synthetic plastics material or a combination of synthetic material and fibres.

In another construction according to the invention the bearing liner can be carried in a second liner in relation to which it can swivel and which is located in fixed or free relationship in the backing to provide a dual mobility cup. This second bearing liner can again be made from metal, synthetic plastics material, a combination of synthetic material and fibres or a ceramic material.

Preferably the radial thickness of the cup or liner is less over the relieved portion than over the part-spherical portion. This assists in determining its flexible qualities.

The construction of the cup or liner and the materials from which it can be made can be as set out U.S. Pat. No. 5,609,646, U.S. Pat. No. 5,879,387, U.S. Pat. No. 6,758,864, U.S. Pat. No. 6,638,311, U.S. Patent Publication No. 2006/0116774 and U.S. patent application Ser. No. 11/406,688 filed Apr. 14, 2006, the disclosure of which is incorporated herein by reference.

Thus, the cup can be made from a combination of PEEK and carbon fiber any other suitable material.

The invention also includes a prosthetic flexible bearing element cup or cup bearing liner in combination with a part-spherical bearing head and such a head can be made from any convenient material, for example metal, a synthetic plastics material, a combination of synthetic material and fibres or a ceramic material, or a metal and ceramics composite.

The head can be rigid with a fixing element for example in the form of a stem or it can be removably attached to such a stem.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

FIG. 1 is a diagrammatic cross-section of a ball head in a flexible acetabular cup according to the invention.

FIGS. 2, 3 and 4 are diagrammatic cross-sections of conventional ball and cup constructions which are used to illustrate how the values a and b are determined for use in the formula employed in the invention.

FIGS. 5-19 are a series of graphs showing the effect of using values a and b at various angles to produce curved relieved portions of different shapes.

### DETAILED DESCRIPTION

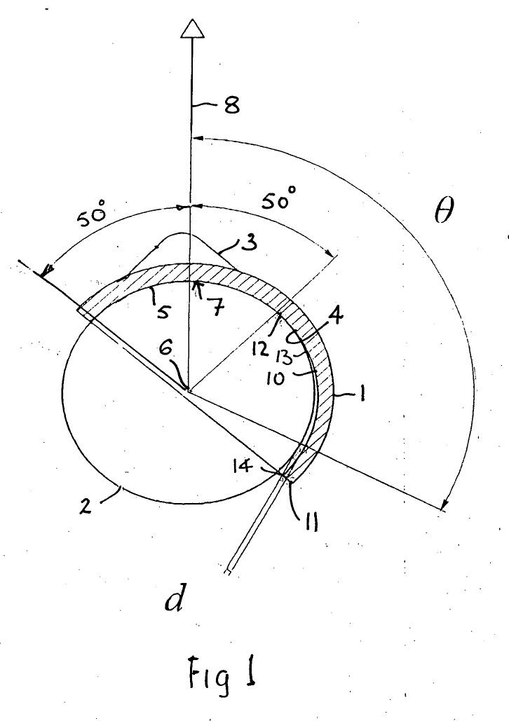

As shown in FIG. 1 the cup to which the invention is to be applied is indicated by reference numeral 1 and is for use with a femoral ball head 2, the cup 1 is of the flexible type and can be shaped as set out in U.S. Pat. No. 5,609,646, U.S. Pat. No. 5,879,387, U.S. Pat. No. 6,758,864, U.S. Pat. No. 6,638,311, and U.S. Patent Publication 2006/0116774.

A fin 3 of the kind shown in U.S. Pat. No. 6,758,864 can be used. The cup 1 has an inner bearing surface 4 which has a first portion 5 which is substantially part-spherical about a hemispherical center 6 and in which there is a polar bearing area 7 which is in line with a main loading axis 8. In the construction shown the part-spherical bearing surface 5 extends through about 50 degrees on each side of the main loading axis 8.

A second portion of inner bearing surface 4 is indicated by reference numeral 10 which extends from main loading axis 8 to a rim 11 and overlaps first portion 5 for about 50°. From this point part of second portion 10 is relieved with respect to first portion 5 and has a radial distance from hemispherical center 6 which increases as the angle of rotation θ about the hemispherical center 6 from main loading axis 8 towards rim 11 of cup 1 is extended.

Between first inner surface portion 5 and the second inner surface portion 10 there is a short transition portion 12 provided by the overlap. The inner end of the relieved part of the second inner surface portion 10 adjacent transition portion 12 is indicated by reference numeral 13 and the outer end adjacent the rim 11 is indicated by reference numeral 14,

As shown in FIG. 1 the radial distances of the surface of second portion 10 from the line representing the shape of the outer surface of part-spherical bearing head 2 when located on main loading axis 8 and on hemispherical center 6 is indicated by reference letter d.

The distance d determines the amount and shape of the relieving at any given angle θ from main loading axis 8.

According to the present invention the distance d is determined by the formula
d=a(e^{θln b}−1)
where
d is the radial distance in millimeters between the line representing said shape of the outer surface of the part-spherical bearing head when located on said first portion of the inner bearing surface and with its center located on said hemispherical center and the second portion,
θ is the angle in degrees about the hemispherical center.

In order to choose the distances d at specific angles θ it is necessary to understand the bearing principals with conventional cups and reference is made to FIGS. 2, 3 and 4 in which the same reference numerals are used to indicated similar parts to those shown in FIG. 1.

When a convention femoral ball head 2 is placed in a conventional cup, indicated by reference numeral 15 in FIG. 2, in order to allow movement of the head in the cup the diameter 16 of the head 2 has to be smaller than the Inner diameter 17 of the cup 15. This is necessary because the surfaces of the head and cup are never perfect. As a consequence of which there is a clearance 18 which is commonly defined as the radial clearance which is:

r radial clearance=radius of the cup−radius of the head

The value of the radial clearance depends on the materials used for the head and the cup. It is usually around 50μ 100 μm for hard bearing couples (metal head into metal cup or ceramic into ceramic cup) and 150 μm to 300 μm for a soft bearing couple (metal or ceramic head into a polyethylene cup). If a composite material is used for the cup which is articulating against a ceramic head, the radial clearance can also be 150 μm to 300 μm.

When a load is applied and the head and the cup are both made of nondeformable material, the head will move along the main loading axis 8 and come into contact with the inner surface of the cup, as shown in FIG. 3. When this occurs there is only one point contact, indicated by reference numeral 20, with the inner surface of the cup 15. The distance d, indicated by reference numeral 18, between the head and the cup is increasing progressively about an angle θ from the main loading axis 8, as shown in FIG. 1.

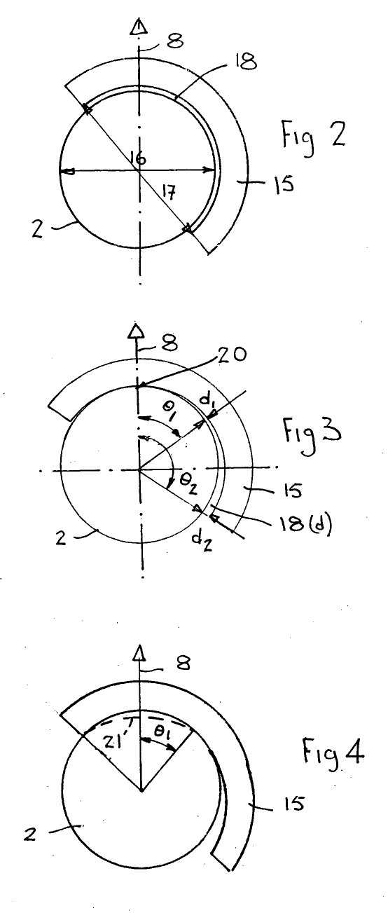

If the head and the cup were considered as being formed from elastic material (usually polyethylene) for the cup and steel for the head then a deformation will happen predominantly on the softer material. The softer the material the higher the deformation under the same loading condition, FIG. 4 shows the deformation of the cup and the broken line 21 shows the shape of the cup prior to the deformation. In this case the contact between the head and the cup forms a sector on which the half-angle is θ₁.

This angle θ₁ can be determined by experimentation or calculation, thus for a couple of bearing materials the angle of the contact sector under defined loading conditions can be predicted. With the flexible cup, the bearing portion will function similarly to a conventional cup. Therefore it is possible to predict the contact sector between the head and the cup and find the angle θ₁ corresponding to the end of the deformation of the softer material. In the example shown in FIG. 1 this angle is 50 degrees. It is now necessary to determine the distance d¹ at the angle θ₁. This is done by returning to the non-deformable conditions shown in FIG. 3 and measuring the distance d¹ at said angle θ₁. This distance can also be easily calculated. In the example shown in FIG. 1 the distance can be 0.09 mm. This means that the curve between θ=0 degrees and θ=50 degrees will be similar to the curve of a conventional cup with a spherical inner cavity.

The second specific angle θ₂, as shown in FIG. 3, is taken at the end of the cup when it is placed in the standard position. At this angle distance d² is again required between the end of the cup adjacent the rim so that it accommodates its predictable deformity of the reamed host bone and also accommodates the natural deformation of the acetabulum during normal gait cycle. These figures come from geometrical analysis of the acetabulum after reaming and strain analysis during the gait.

In the example shown in FIG. 3 the distance d² at the angle θ₂ is 1.2 mm. Thus, the curvature generated with the function would pass through these two specific points defined.

If the first angle θ₁ is taken at 50 degrees and the second angle θ₂ at 130 degrees and the distance d¹ at the first angle is 0.09 mm and the distance d₂ at 1.2 mm then the two equations are set out as follows:
0.09=a(e^{50 ln b}−1) and 1.20=a(e^{130 ln b}−1)

From the two equations it is possible to determine the following values for a and b:
a≈0.02689425 and b=1.0298232

The equation of the relieving curve is then established and can be written
d=0.02689425(e^{θ ln 1.0298232}−1)

The distance d₁ can be between 0.050 mm to 0.500 mm and the distance d₂ can be between 0.400 mm and 5,000 mm.

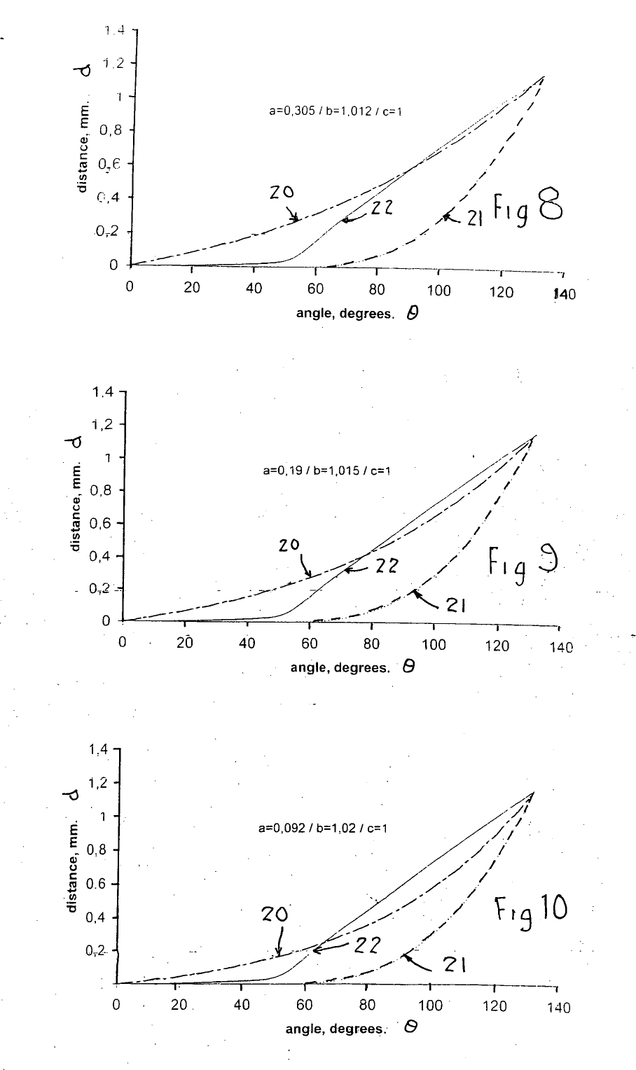

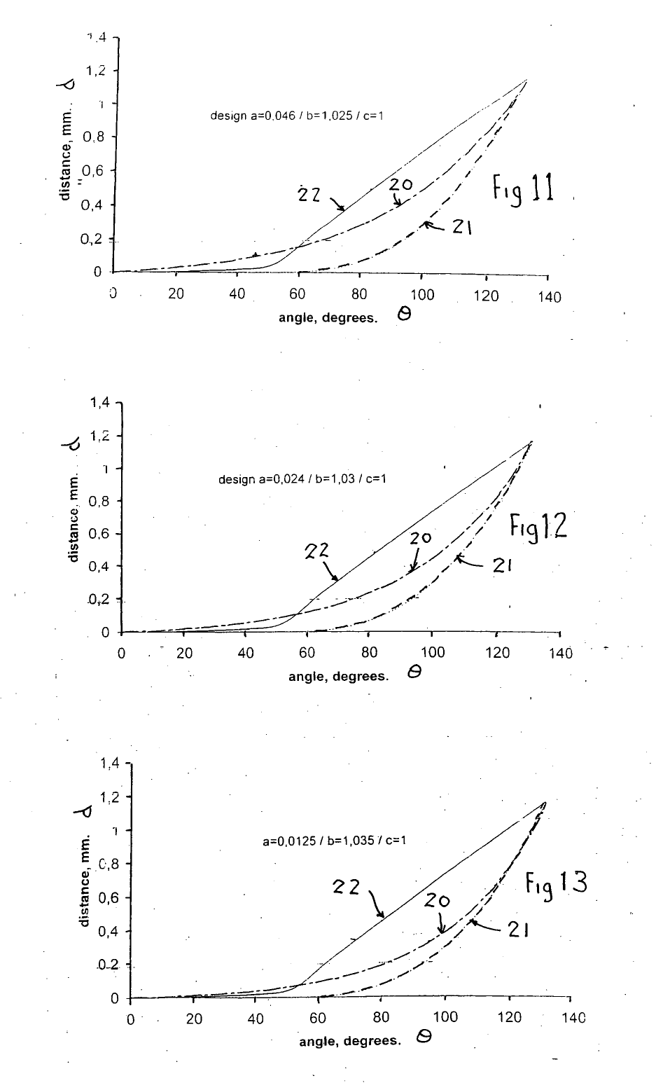

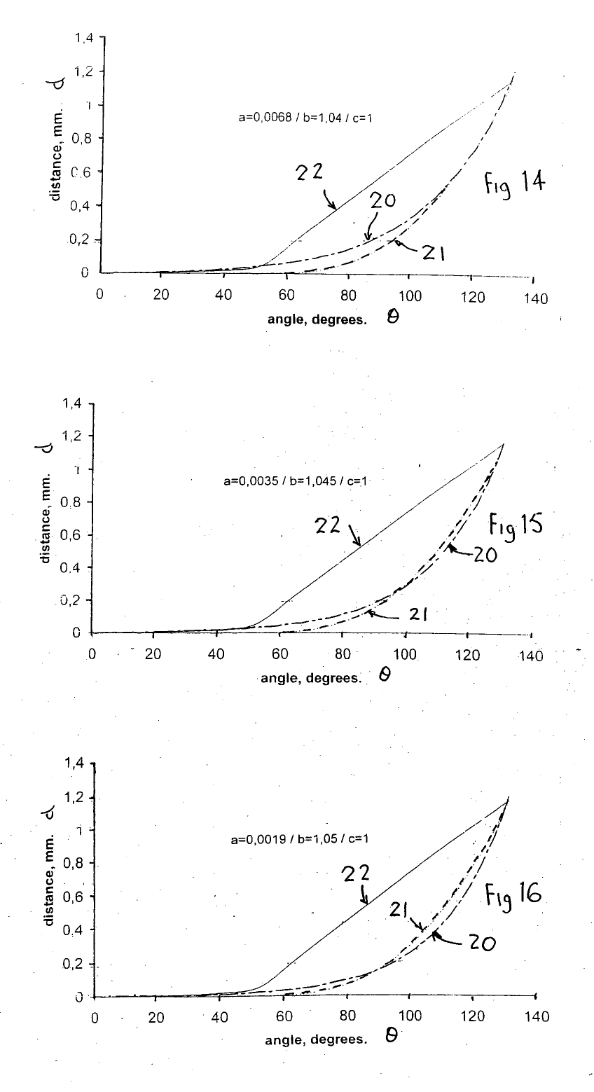

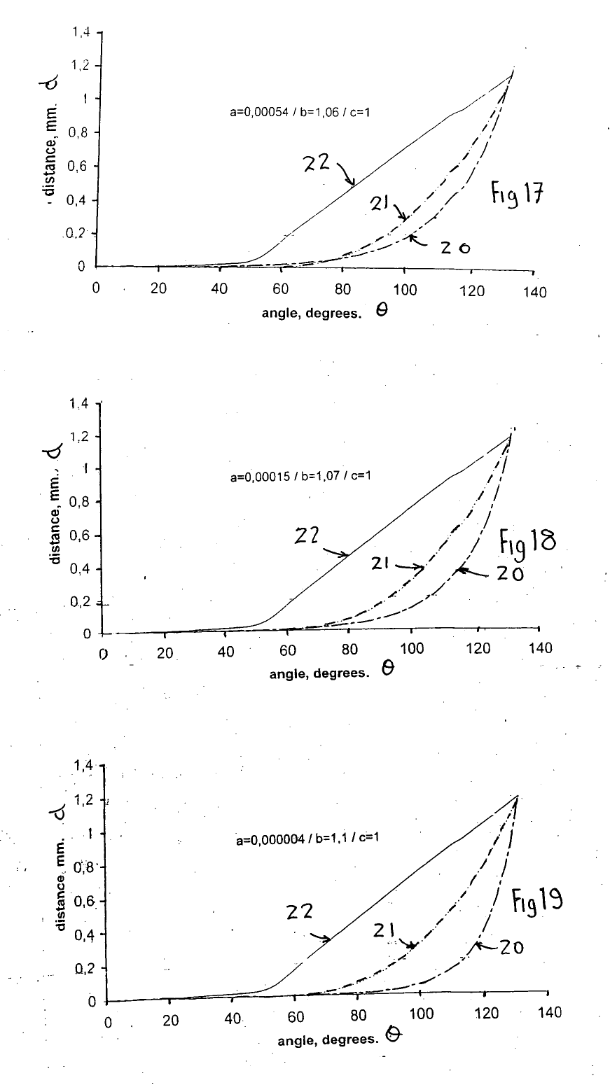

The graphs shown in FIGS. 5 to 19 show the distance d in millimetres for the determined degree θ from the main loading axis 8.

In the graph shown in FIG. 5 the a and b values are indicated and the broken chain line 20 shows the distance d for the various angles θ. It will be seen that the line is substantially straight and the relieving commences from the main loading axis 8. This type of relieving is suitable for use with flexible cups which are relatively stiff. In this arrangement the first part-spherical portion 5 only extends over a curve of about 50 degrees to one side of the main bearing axis 2.

For comparison the graph also shows the distance d for various angles θ for a construction made according to the information set out in US 2005/0256584 and this is indicated by broken line 21 and solid line 22 shows the distances for relieving as set out in U.S. Pat. No. 5,609,646.

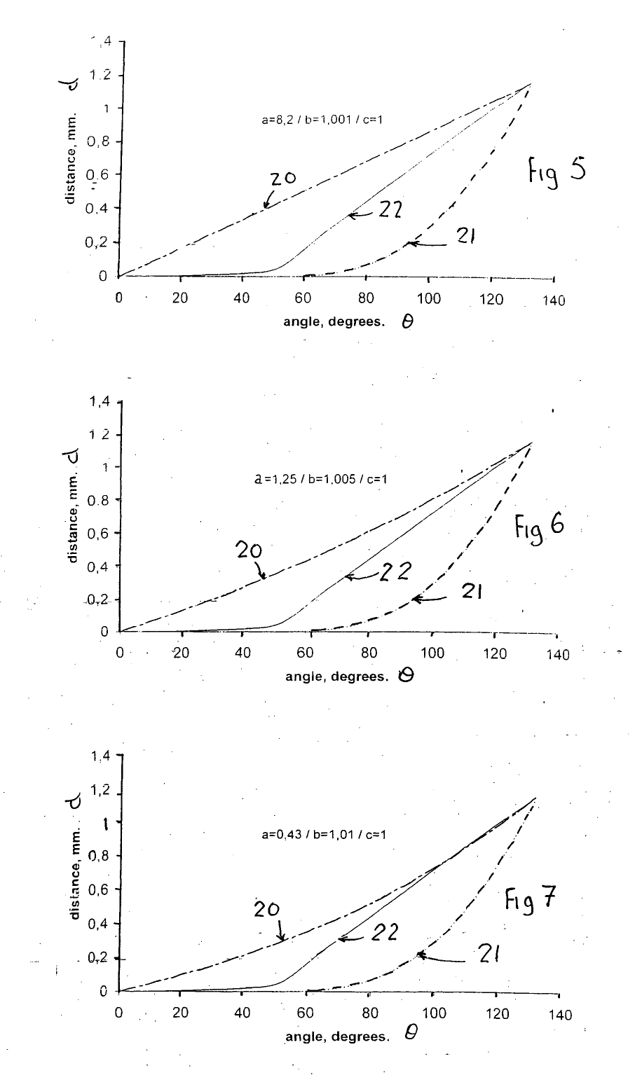

Graphs shown in FIGS. 6, 7 and 8 show variations on the shape as compared with the graph shown in FIG. 5 and in FIG. 9 the dimensions produced by the formula still provide for the commencement of relieving in the area of the main bearing axis 8.

From the graphs of FIGS. 10 to 13 the dimensions become smaller as the angle moves away from the main bearing axis 8 until the graph of FIG. 14 where it will be seen that there is little movement until about 50 degrees from the main bearing angle. The movement then is gradual so that there is, in effect, a transition portion between about 50 degrees and 60 degrees.

From the graphs of FIGS. 15 to 19 there is again only a small amount of movement and from the graph of FIG. 19 there is little movement away from the part-spherical surface of the first portion of the inner bearing surface until the angle is about 80 degrees.

The later curves shown from about FIGS. 14 to 19 are more suitable for cups which are more flexible.

The invention is particularly designed to produce cups in which a particular effect is required to reduce the possibility of seizure or heavy wear.

The invention also includes a method of making a prosthetic cup of the kind as set forth.

The values obtained with the function described above in the present invention need to be added to the femoral head radius in order to generate the inner shape of the cup. This is a basic transformation done with convention Computer Added Design (CAD) software, as well as the transformation of the function in a series of co-ordinates of points that are used to program a Computerised Numerical Control (CNC) lathe. The transformation usually needs to tolerate an approximation between the theoretical curve and actual curve that is used for the machining. A 1 μm approximation allows the generation of a curve with an acceptable number of points.

The construction of an acetabular cup including an inner portion following the curve described in the present invention requires to position the inner portion relative to the outer shape of the cup. The objective is to maintain the center of rotation of the prosthetic head as close as possible to the natural center of rotation, therefore, the center of the femoral head is usually close to the center of the outer part-spherical portion of the cup. The application of this rule to the present invention will result in an acetabular prosthesis with a variable cup wall thickness. In the present construction the thickest wall is preferably located in the bearing portion and the thinner wall is positioned near the opened portion of the cup. This thickness distribution allows the stiffening of the portion where deformation needs to be avoided and soften the portion where deformation is expected.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic flexible bearing element cup or cup bearing liner adapted for use with a part-spherical bearing head comprising an inner bearing surface which has a first portion which is substantially part-spherical about a hemispherical center located on a main loading axis, and a second portion at least part of which is relieved with respect to the first portion and which over said relieved part has a radial distance (d) from the hemispherical center which increases from a inner end towards an outer end as the angle of rotation (θ) about the hemispherical center increases towards the rim of the cup, wherein the distance of the relieved part of the surface of the second portion from a line representing the shape of the outer surface of the part-spherical bearing head when located on said main loading axis and bearing against said first portion of the inner bearing surface at any given radial distance (d) is determined by the formula
d=a(e^{θ ln b}−1)
where
D is the radial distance in millimeters between the line representing said shape of the outer surface of the part-spherical bearing head when located on said first portion of the inner bearing surface and with its center located on said hemispherical center and the relieved part of the second portion
θ is the angle in degrees about the hemispherical center, and
a and b are values determined by selecting angle an (θ₁) and a distance (d₁) at or towards said inner end of the second portion and placing them in said formula, selecting an angle (θ₂) and a distance (d₂ at or towards the outer end of second portion adjacent the rim and placing them in the formula and solving the two equations together.

2. The prosthetic flexible bearing element cup or cup bearing liner as claimed in claim 1 wherein the values of a and b are selected from the following Table:

| a | b |
|---|---|
| 8.2 | 1.001 |
| 1.25 | 1.005 |
| 0.43 | 1.01 |
| 0.305 | 1.012 |
| 0.19 | 1.015 |
| 0.092 | 1.02 |
| 0.046 | 1.025 |
| 0.024 | 1.03 |
| 0.0125 | 1.035 |
| 0.0068 | 1.04 |
| 0.00035 | 1.045 |
| 0.0019 | 1.05 |
| 0.00054 | 1.06 |
| 0.00015 | 1.07 |
| 0.000004 | 1.1 |

3. The prosthetic flexible bearing element cup or cup bearing liner as claimed in claim 1 wherein the second portion extends from the main loading axis to the end thereof adjacent its rim.

4. The prosthetic flexible bearing element cup or cup bearing liner as claimed in claim 1 wherein a transition portion of the inner bearing surface between the first portion and the relieved part of the second portion is provided.

5. The prosthetic flexible bearing element cup or cup bearing liner as claimed in claim 4 wherein the end of the second portion overlaps the first portion up to the main loading axis, to provide said transition portion.

6. The prosthetic flexible bearing element cup or cup bearing liner as claimed in claim 1 wherein the inner bearing surface is formed in a one-piece cup.

7. The prosthetic flexible bearing element cup or cup bearing liner as claimed in claim 6 wherein the one-piece cup is made from metal, a synthetic plastics material, a combination of synthetic materials and fibers, or a ceramic material.

8. The prosthetic flexible bearing element cup or cup bearing liner as claimed in claim 1 wherein the inner bearing surface is formed on a bearing liner carried in a backing.

9. The prosthetic flexible bearing element cup or cup bearing liner as claimed in claim 8 wherein the liner is made from metal, a synthetic plastics material, a combination of synthetic material and fibers or a ceramic material.

10. The prosthetic flexible bearing element cup or cup bearing liner as claimed in claim 8 wherein the backing is made from metal, synthetic plastics material or a combination of synthetic material and fibers.

11. The prosthetic flexible bearing element cup or cup bearing liner as claimed in claim 8 wherein the bearing liner is carried in a second liner in relation to which it can swivel and which is located in fixed or free relationship in the backing to provide a dual mobility cup.

12. A prosthetic flexible bearing element cup or cup bearing liner as claimed in claim 11 wherein the second bearing liner is made from metal, synthetic plastics material, a combination of synthetic material and fibers or a ceramic material.

13. The prosthetic flexible bearing element cup or cup bearing liner as claimed in claim 1 wherein the radial thickness of the cup or liner is less over the relieved portion than over the part-spherical portion.

14. The prosthetic flexible bearing element cup or cup bearing liner as claimed in claim 1 wherein the cup is made from a combination of PEEK and carbon fiber.

15. The prosthetic flexible bearing element cup or cup bearing liner as claimed in claim 1 in combination with a part-spherical bearing head.

16. The prosthetic flexible bearing element cup or cup bearing liner as claimed in claim 15 in combination with a part-spherical bearing head which is made from metal, a synthetic plastics material, a combination of synthetic material and fibers or a ceramic material, or a metal and ceramics composite.

17. The prosthetic flexible bearing element cup or cup bearing liner in combination with a head as claimed in claim 15 wherein the head is rigid with a fixing element.

18. The prosthetic flexible bearing element cup or cup bearing liner as claimed in claim 17 wherein the fixing element is in the form of a stem or it can be removably attached to such a stem.

19. The prosthetic flexible bearing element cup or cup bearing liner as claimed in claim 16 in which the head is removably attached to a stem.

20. A prosthetic flexible acetabular cup bearing or bearing liner comprising:
a bearing component having a curved inner bearing surface, the bearing surface having a main loading axis extending through a center of a part-spherical surface;
a distance d between the part-spherical surface and the curved inner bearing surface of the bearing component increasing as the angle between the main loading axis and a radius through the center of the part-spherical surface increases, d being defined by the formula
d=a(e^{θ ln b}−1)
where
D is the radial distance in millimeters between the line representing said shape of the outer surface of the part-spherical bearing head when located on said first portion of the inner bearing surface and with its center located on said hemispherical center and the relieved part of the second portion
θ is the angle in degrees about the hemispherical center, and
a and b are values determined by selecting angle an (θ₁) and a distance (d₁) at or towards said inner end of the second portion and placing them in said formula, selecting an angle (θ₂) and a distance (d₂ at or towards the outer end of second portion adjacent the rim and placing them in the formula and solving the two equations together.

21. The prosthetic flexible acetabular cup bearing or bearing liner as set forth in claim 20 wherein the main loading axis passes through the center of the part-spherical surface.

22. The prosthetic flexible bearing element cup or cup bearing liner as claimed in claim 21 wherein the values of a and b are selected from the following Table:

| a | b |
|---|---|
| 8.2 | 1.001 |
| 1.25 | 1.005 |
| 0.43 | 1.01 |
| 0.305 | 1.012 |
| 0.19 | 1.015 |
| 0.092 | 1.02 |
| 0.046 | 1.025 |
| 0.024 | 1.03 |
| 0.0125 | 1.035 |
| 0.0068 | 1.04 |
| 0.00035 | 1.045 |
| 0.0019 | 1.05 |
| 0.00054 | 1.06 |
| 0.00015 | 1.07 |
| 0.000004 | 1.1 |
